(19) United States
(12) Patent Application Publication
(10) Pub. No.: US 2025/0169464 A1
(43) Pub. Date: May 29, 2025

(54) Animal Bedding with Status Indicator

(71) Applicant: Allentown, LLC, Allentown, NJ (US)

(72) Inventors: Casey Bonagura, Ocean Grove, NJ (US); Steve Miller, Allentown, NJ (US)

(21) Appl. No.: 18/962,584

(22) Filed: Nov. 27, 2024

Related U.S. Application Data
(60) Provisional application No. 63/603,553, filed on Nov. 28, 2023.

(51) Int. Cl. A01K 1/015; G01N 33/52

(52) U.S. Cl. CPC A01K 1/0154; A01K 1/0155; G01N 33/528

(57) ABSTRACT

Small animal bedding comprising an indicator feature that responds to specific biomarkers in mouse urine and fecal matter and methods for making the same. The indicator feature can change color upon detection of one or more biomarkers. Optionally, an indicator feature can change color upon detection of threshold level of ammonia adjacent to the bedding.

### BACKGROUND OF THE INVENTION

### Field of the Invention

The present invention relates in general to animal bedding and, in particular, to animal bedding including a status indicator for monitoring health or environment of the animal.

### Description of Related Art

Conventional small animal bedding can be formed of paper fibers. US Patent Publication Number 20070012259 describes a cellulose particulate provided in bright white or in vivid colors. Brightness and color clarity are maintained during manufacture by apparatus having non-corroding surfaces.

In the realm of laboratory animal science, a primary concern is the monitoring of the health and well-being of research animals. It is desirable to provide improved animal bedding including an indicator for monitoring the health of the animal to benefit research, husbandry practices and animal welfare.

### BRIEF SUMMARY OF THE INVENTION

The present invention relates to small animal bedding comprising an indicator feature that responds to specific biomarkers in mouse urine and fecal matter and methods for making the same. In one embodiment, the small animal bedding can be used for rodents, such as for example mice and rats. Urinary and fecal biomarkers can be used as potential markers for disease. Example biomarkers can include indicators of stress, infection, or dehydration. The indicator feature can be a change of color of the bedding.

In one embodiment, if there is a high level of alkaline in urine excreted from an animal onto the bedding, the indicator feature of the bedding will change color. The change of color of the indicator feature of the bedding provides an early visual indication of potential health abnormalities of the animal.

In one embodiment, the indicator feature of the bedding can be an ammonia sensitive component. The indicator feature will change color when an ammonia level adjacent to the bedding reaches a predetermined threshold. For example, the bedding will change color when ammonia levels exceed the threshold, signaling the need for a bedding change.

### BRIEF DESCRIPTION OF THE SEVERAL VIEWS OF THE DRAWING(S)

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a schematic diagram of animal bedding with status indicator.

### DETAILED DESCRIPTION OF THE INVENTION

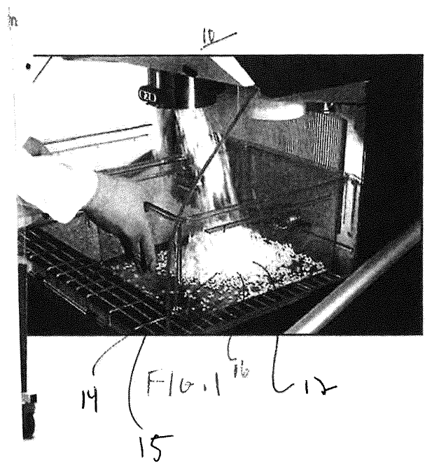

FIG. 1 is a schematic diagram of animal bedding with status indicator 10. Animal bedding 12 can be formed of fibrous or particulate material. Suitable materials include lignocellulosic fibers derived from wood-based materials, cellulose fiber paper, shredded paper, strips, pieces of paper and paper bits. Suitable animal bedding can be manufactured according to US Patent Publication Number 20070012259, hereby incorporated by reference into this application. Animal bedding 12 can be used for small animals. Small animals can comprise rodents, such as for example mice and rats.

Indicator 14 can be associated or integral with animal bedding 12. For example, indicator 14 can be combined during formation of animal bedding 12. In one embodiment, indicator 14 can be coated or sprayed on animal bedding 12. In one embodiment, indicator 14 can comprise indicator material 15 which is separate from animal bedding 12 and is combined with animal bedding 12. Indicator material 15 can comprise fibrous or particulate material. Animal bedding 12 can comprise fibrous or particulate material. The fibrous or particulate material of indicator material 15 can be combined with the fibrous or particulate material of animal bedding 12. Animal bedding 12 can be used as an animal litter.

Indicator 14 can comprise one or more urinary and/or fecal biomarkers. Example biomarkers can include indicators of stress, infection, or dehydration. Indicator 14 can change color due to the presence or absence of one or more urinary or fecal biomarkers. Example urinary biomarkers found in mouse urine include albumin, kidney injury molecule-1 (KIM-1), neutrophil gelatinase-associated lipocalin (NGAL), osteopontin (OPN), cystatin C, N-acetyl-β-d-glucosaminidase (NAG), and beta-2 microglobulin (B2M). Example fecal biomarkers include fecal volatile organic compounds.

Ammonia indicator 16 can comprise an ammonia sensitive component. Ammonia indicator 16 can change color when an ammonia level adjacent to animal bedding 12 reaches a predetermined threshold. Ammonia indicator 16 can optionally be used with indicator 14. Ammonia indicator 16 can comprise a pH sensitive indicator. Example materials for ammonia indicator 16 include phenolphthalein and bromothymol blue.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. Animal bedding with status indicator comprising:
animal bedding; and
the status indicator being associated or integral with the animal bedding, wherein the status indicator detects presence or absence of one or more urinary or fecal biomarkers.

2. The animal bedding according to claim 1, wherein the status indicator changes color to in the presence or absence of one or more urinary or fecal biomarkers.

3. The animal bedding according to claim 1, wherein the status indicator is coated or sprayed on the animal bedding.

4. The animal bedding according to claim 1, wherein the status indicator is an indicator material which is separate from the animal bedding.

5. The animal bedding according to claim 4, wherein the indicator material comprises fibrous or particulate material.

6. The animal bedding according to claim 1 wherein the animal bedding comprises fibrous or particulate material.

7. The animal bedding according to claim 1, wherein the bedding material is one or more materials of lignocellulosic fibers derived from wood-based materials, cellulose fiber paper, shredded paper, strips, pieces of paper of paper bits.

8. The animal bedding according to claim 1, wherein comprise one or more urinary and/or fecal biomarkers.

9. The animal bedding according to claim 1, wherein the one or more urinary and/or fecal biomarkers include indicators of stress, infection, or dehydration.

10. The animal bedding according to claim 1, wherein the one or more urinary biomarkers are one or more of albumin, kidney injury molecule-1 (KIM-1), neutrophil gelatinase-associated lipocalin (NGAL), osteopontin (OPN), cystatin C, N-acetyl-β-d-glucosaminidase (NAG) or beta-2 microglobulin (B2M).

11. The animal bedding according to claim 1, wherein fecal biomarkers comprise fecal volatile organic compounds.

12. The animal bedding according to claim 1 further comprising an ammonia indicator.

13. The animal bedding according to claim 12 wherein the ammonia indicator changes color when an ammonia level adjacent to the animal bedding reaches a predetermined threshold.

14. The animal bedding according to claim 11 wherein the ammonia indicator comprises a pH sensitive indicator.

15. The animal bedding according to claim 14 wherein the pH sensitive indicator comprises phenolphthalein of bromothymol blue.

16. A method for manufacturing animal bedding with status indicator comprising the step of:
applying the status indicator to the animal bedding, wherein the status indicator detects presence or absence of one or more urinary or fecal biomarkers.

17. The method of claim 16 wherein the applying step comprising spraying or coating the status indicator on the animal bedding.

18. A method for manufacturing animal bedding with status indicator comprising the step of: combining a fibrous or particulate material of the animal bedding with a fibrous or particulate material of the status indicator, wherein the status indicator detects presence or absence of one or more urinary or fecal biomarkers.

19. The method of claim 18 further comprising the step of: combining an ammonia indicator with the animal bedding.

20. The method of claim 19 wherein the ammonia indicator changes color when an ammonia level adjacent to the animal bedding reaches a predetermined threshold.
